(19) United States
(12) Patent Application Publication
(10) Pub. No.: US 2001/0005721 A1
(43) Pub. Date: Jun. 28, 2001

(54) Heel crack healing composition and method therefor

(76) Inventor: Fereidoon Pourbastani, Gilbert, AZ (US)

Correspondence Address: MARTIN L. STONEMAN, 3113 North 3rd Street, Phoenix, AZ, 85012, US

(21) Appl. No.: 09/772,006

(22) Filed: Jan. 29, 2001

Related U.S. Application Data
(63) Continuation-in-part of application No. 09/329,961, filed on Jun. 9, 1999, now Pat. No. 6,173,984.
Application No. 09/329,961 is a continuation-in-part of application No. 09/065,218, filed on Apr. 23, 1998, now abandoned.

(51) Int. Cl. A61K 31/56

(52) U.S. Cl. 514/164; 514/171

(57) ABSTRACT

Described is a method and heel cream composition for healing epidermal cracks on the soles of feet, primarily on heels, having active ingredients consisting of a combination of a safe and effective amount of salicylic acid and hydrocortisone in some preferred embodiments, with further embodiments further consisting of salicylic acid, hydrocortisone, boric acid, and zinc oxide. The preferred embodiments also comprise a preferred carrier for transporting the active ingredients to the heel cracks. The method of preparation includes, using a specified heat, separately mixing an oil phase, a water phase, and a medicate phase (preferably including the salicylic acid and the hydrocortisone) and then combining them. As a method of treatment, the heel cream composition should be topically applied to the skin areas having epidermal cracks about twice a day.

### CROSS REFERENCE TO RELATED APPLICATIONS

The present application is a continuation-in-part of U.S. application Ser. No. 09/329,961 filed Sep. 09, 1999 now abandoned, which was a continuation-in-part of U.S. application Ser. No. 09/065,218 filed Apr. 23, 1998 previously abandoned.

### BACKGROUND

This invention relates to the field of non-prescription topical compositions for treatment of epidermal cracks on the sole of the foot, specifically cracks in the heel. More specifically, this invention relates to a skin care composition in the form of a heel cream for providing complete relief and healing from the condition characterized by cracks in the heels and soles of feet, as well as the associated methods. Typically, consumers have long desired moisturizing lotions and creams that both benefit the skin and provide cosmetically acceptable tactile properties. Typically, many of these moisturizers have contained ammonium salts or mineral oils, and even more often, contained petrolatum. Examples of these formulations can be found in U.S. Pat. Nos. 4,454,188 and 4,883,659 issued to, respectively, Johnson and Goodman et al. But theorists in the field have observed that creating a moisturizer is still a trial-and-error art resting on a weak scientific foundation, and further that rational principles alone will not produce a superior moisturizer unless aesthetics, etc., are ignored.

More particularly, there has been a dearth of moisturizers suitable for the soles of the feet, and even more particularly, the heels of the feet. It is well known that many persons often suffer from heels that are dry and cracked, an embarrassing and often painful condition characterized by the formation of severe epidermal cracks in the heel. And there have been few, if any, available treatments or available preparations for application to such areas to assist such persons in healing such cracks. For this purpose, in addition to moisturizing qualities, there have not been available, for example, heel creams which provide relief and healing from the condition by including properties such as softening and immune suppression, as well as germicidal properties, etc.

Prior art formulations which have developed to assist in the healing and prevention of less problematic skin wrinkles can be found in U.S. Pat. No. 5,744,148 issued to Habif et al., and the above-mentioned U.S. Pat. No. 4,454,118 issued to Johnson. These two patents teach skin care compositions used to provide relief from wrinkles, chapped skin, or other similar common skin conditions. Though these formulations may be adequate for their respective intended purposes, neither teaches a composition that provides a healing and relief from the more severe heel cracks and fissures that are oftentimes painful, embarrassing, and debilitating. In addition, neither teaches an associated treatment method using their respective compositions for specifically treating heel cracks. Addressing yet another patent, U.S. Pat. No. 5,691,327 issued to Blank, Blank discloses that formulations to treat wrinkles may contain, among other possible ingredients, salicylic acid, hydrocortisone, zinc oxide and boric acid in concentration ranges of from 0.1% to 20%. But Blank does not teach nor enable a heel crack healing composition nor teach the specific ranges of respective ingredients that have testably been most therapeutic (e.g., the preferred embodiments of the present invention).

It is noted that zinc oxide was included by Blank as a sun screen agent (sun screen agents would not be required on the heel of the foot). Blank further discloses the use of salicylic acid as one possible ingredient in solutions, lotions, creams, beach products, gels, sticks, sprays, pads, ointments, pastes, mousses and cosmetics (12 possible forms). Blank also lists pharmaceutically-acceptable organic solvents which might include propylene glycol, butylene glycol, polyethylene glycol, polypropylene glycol, glycerol, 1,2,4 botanetriol, sorbitol esters, 1,2,6 hexanetriol, ethanol, isopropanol, butanediol, and mixtures thereof (11 possible ingredients, Column 4, lines 26-31). Blank lists a sun screen element which may be included which may consist of p-aminobenzoic acid, anthranilates, salicylates, cinnamic acids, dihydroxycinnamic acids, trihydroxycinnamic acids, hydrocarbons, dibenzalacetone and benzalacetophenone, naphtholsulfonates, dihydroxynaphthoic acids, coumarin, diazoles, quinine salts, quinoline derivatives, hydroxy-or methoxy-substituted benzopheonones, uric and vilouric acids, tannic acids, ether, hydroquinone, benzophenones, etc. in safe and effective concentrations (71 possible ingredients). Blank's list also includes anti-inflammatory agents (60 possible ingredients), anti-oxidants (13 possible ingredients), chelators (2 possible ingredients), retinoids (2 possible ingredients), Benzofuran derivatives (included or excluded adds 2 possibilities), N-acetyl-L-cysteine derivatives (44 possible ingredients), and others. The above incomplete list of Blank's ingredients does not include vitamins, secondary anti-inflammatory agents, or skin protectants also therein discussed. This incomplete list also does not include ranges of concentrations of these possible ingredients which adds infinite variety to the above list. Thus, even though incomplete, this list describes more than 2.5 billion different compounds. To state that Blank, with its 2.5 billion possible compounds, places any one of those possible compounds in the possession of the public for heel cracking, without clinical testing to discover, e.g., what are the clinically preferred therapeutic compounds, would not be proper.

In contrast, the preferred embodiments of the current invention describe significant clinical trials that were conducted to determine the clinically effective composition of active ingredients, the clinically effective concentrations of those active ingredients, and the clinically effective method of use of the resulting composition. Thus, the preferred embodiments of the present invention are the result of long periods of clinical testing to maximize the clinical effectiveness of this invention. The significant effectiveness of these tested compositions in treating heel cracks yield a surprising result modified and maximized through clinical trials.

Again, Blank does not even mention foot or heel cracks. Heel cracks are different from calluses, corns, warts, eczema, wrinkles, the thinning of the skin which comes from age, dry skin, chaffed skin, chapped skin or any other skin condition. The only common characteristic shared by these conditions is that they all occur in skin. Skin conditions vary widely and can be treated in widely variable ways. More specifically, calluses are thickened and hardened areas on skin, sometimes feet, caused by continuous rubbing against skin. Calluses are useful in preventing injury to the skin in areas of constant rubbing and use. Calluses can be left untreated with no adverse consequences. Corns are defined in the Random House Dictionary as horny indurations or callosities of the epidermis, usually with a central core, formed especially on the toes or feet and caused by undue pressure or friction. Corns are sometimes treated by surgical means. Warts are viral in origin and can occur anywhere on the body. Warts are treated surgically or with highly concentrated solutions of salicylic acid. Wrinkles, the focus of the Blank patent, are a natural result of age and exposure to sun and weather. Wrinkles can be left untreated, but may be treated for cosmetic reasons.

In contrast, foot cracks, and more specifically, heel cracks, the malady with which preferred embodiments of the present invention deals, are a condition of sometimes deep, painful, itchy, irritating, embarrassing, fissures in the skin of the feet, most often the heels of the feet. Heel cracks are not associated with warts, corns or wrinkles. Heel cracks can cause pain, making walking painful. Heel cracks can be a site of infection if the fissures are deep. Heel cracks can be associated with calluses. Blank's disclosure gives the public no indications of which of the 2.5 billion compounds might be most effective in treating heel cracks. Blank's disclosure gives no indications of what standard might be critical in developing a compound to treat sole cracks nor more specifically heel cracks, nor even where to begin experimentation. The results obtained by embodiments of the present invention are surprising results of curing heel cracks with certain described methods and compositions. These surprising results were maximized with long periods of clinical trials and testing.

### OBJECTS OF THE INVENTION

A primary object of the present invention is to fulfill the above-mentioned needs by the provision of a heel cream for use in healing epidermal cracks on the soles of the feet, particularly heel cracks. A further primary object of the present invention is to provide such a heel cream which overcomes the prior deficiencies and is inexpensive and easy to apply. Other objects of this invention will become apparent with reference to the following invention description.

### SUMMARY OF THE INVENTION

According to a preferred embodiment of the present invention, this invention provides a method for healing epidermal cracks in heels comprising the acts of topically applying to the affected skin areas having such epidermal cracks a composition having a safe and effective amount of active ingredients consisting essentially of salicylic acid, hydrocortisone, and zinc oxide, mixed with a suitable cream mixture carrier; applying such composition to the affected skin areas at least once daily to treat heel cracks; and removing the dead skin surrounding the epidermal cracks with an abrasive material. This invention also provides such a method wherein such active ingredients of such composition are consisting essentially of: from about 2 percent to about 10 percent by weight of such composition of salicylic acid; from about 0.25 percent to about 1 percent by weight of such composition of hydrocortisone; from about 1 percent to about 4 percent by weight of such composition of boric acid; and from about 1 percent to about 7 percent by weight of such composition of zinc oxide. Additionally, this invention also provides such a method wherein such active ingredients of such composition are consisting essentially of: about 4 percent by weight of such composition of salicylic acid; about 0.5 percent by weight of such composition of hydrocortisone; about 2 percent by weight of such composition of boric acid; and about 3 percent by weight of such composition of zinc oxide. Moreover, this invention also provides such a method wherein such application of such composition is done at least twice daily until the epidermal cracks heal. Also, this invention also provides such a method further comprising the subsequent step of applying such composition to the affected skin areas at least once daily to maintain the heel in a healed condition. Further, this invention also provides such a method wherein such abrasive material comprises a pumice stone. And yet further, this invention also provides such a method wherein such abrasive material comprises fine grit sandpaper.

According to another preferred embodiment of the present invention, this invention provides a composition, for treating and healing epidermal cracks on heels, consisting essentially of the following ingredients in the following amounts, by approximate weight percentage of such composition:

| Ingredients | Weight Percent |
|---|---|
| Mineral oil & lanolin alcohol | 8 percent |
| Stearyl alcohol & ceteareth-20 | 5 percent |
| Petrolatum & lanolin & lanolin alcohol | 4 percent |
| Mineral oil | 4.5 percent |
| Glyceryl stearate | 2 percent |
| Vitamin E | 3 percent |
| Fatty alcohol/polysorbate blend | 2 percent |
| Propylene glycol | 4.5 percent |
| Preservative | 1 percent |
| Boric acid | 2 percent |
| Salicylic acid | 4 percent |
| Hydrocortisone | 0.5 percent |
| Zinc Oxide | 3 percent |
| Water | 56.5 percent |

This invention also provides such a composition wherein an application of such composition is done at least twice daily until the epidermal heel cracks heal, and further comprises the subsequent step of applying such composition to the affected skin areas at least once daily to maintain the heel in a healed condition.

According to a further preferred embodiment of the present invention, this invention provides a method for healing epidermal cracks in heels comprising the acts of: topically applying to the affected skin areas having such epidermal cracks a composition having a safe and effective amount of active ingredients consisting essentially of salicylic acid and hydrocortisone, mixed with a suitable cream mixture carrier; applying such composition to the affected skin areas at least once daily to treat heel cracks; removing the dead skin surrounding the epidermal cracks with an abrasive material; and wherein such suitable cream mixture carrier is a member of the class of carriers having properties substantially comparable to the carrier properties of Vaseline and lanolin. This invention also provides such a method wherein such active ingredients of such composition are consisting essentially of: from about 2 percent to about 10 percent by weight of such composition of salicylic acid; and from about 0.25 percent to about 1 percent by weight of such composition of hydrocortisone. Additionally, this invention also provides such a method wherein such active ingredients of such composition are consisting essentially of: about 4 percent by weight of such composition of salicylic acid; and about 0.5 percent by weight of such composition of hydrocortisone. Further, this invention also provides such a method wherein such application of such composition is done at least twice daily until the epidermal heel cracks heal. Moreover, this invention also provides such a method further comprising the subsequent step of applying such composition to the affected skin areas at least once daily to maintain the heel in a healed condition. This invention also provides such a method wherein such abrasive material comprises a pumice stone. An further, this invention also provides such a method wherein such abrasive material comprises fine grit sandpaper.

According to a further preferred embodiment of the present invention, this invention provides a composition, for treating and healing epidermal cracks on heels, consisting essentially of the following ingredients in the following amounts, by approximate weight percentage of such composition:

| Ingredients | Weight Percent |
|---|---|
| Mineral oil & lanolin alcohol | 8 percent |
| Stearyl alcohol & ceteareth-20 | 5 percent |
| Petrolatum & lanolin & lanolin alcohol | 4 percent |
| Mineral oil | 4.5 percent |
| Glyceryl stearate | 2 percent |
| Vitamin E | 3 percent |
| Fatty alcohol/polysorbate blend | 2 percent |
| Propylene glycol | 4.5 percent |
| Preservative | 1 percent |
| Salicylic acid | 4 percent |
| Hydrocortisone | 0.5 percent |
| Water | 56.5 percent |

Also, this invention provides such a composition wherein an application of such composition is done at least twice daily until the epidermal heel cracks heal. This invention also provides such a composition further comprising the subsequent step of applying such composition to the affected skin areas at least once daily to maintain the heel in a healed condition.

### DETAILED DESCRIPTION OF THE PREFERRED EMBODIMENTS AND THE BEST MODE OF PRACTICE

It has been found that relief from cracks which can form on the soles of feet, particularly heels, may be had by topically applying to the affected areas a composition containing, preferably, a safe and effective amount of: salicylic acid powder and hydrocortisone as the active ingredients made and applied in accordance with the teachings herein. Additionally, it has been found that relief from cracks which can form on the heels of feet, may also be had by topically applying to the affected areas a composition containing, preferably, a safe and effective amount of: salicylic acid powder, hydrocortisone, boric acid, and zinc oxide as the active ingredients made and applied in accordance with the teachings herein.

In addition to the active ingredients, it is preferred that the composition also comprise emollients such as petrolatum and lanolin; humectants such as propylene glycol; water as a moisturizer; and emulsifiers such as glyceryl stearate and a fatty alcohol/polysorbate blend. In addition, it is preferred that an appropriate preservative such as Germaben 11 (available from Sutton Laboratories, New Jersey) be used. As used herein, the term “safe and effective amount” refers to that amount of active ingredient(s) necessary to provide the most beneficial results in the shortest amount of time without producing, in accordance with sound and reasoned medical judgment, deleterious side effects.

I) Preferred Composition

Effective results were obtained with the active ingredients preferably comprising between about 2.25 percent to about 11 percent by weight of the composition, with an acceptable range of the salicylic acid component preferably being between about 2 percent and about 10 percent by weight, more preferably about 4 percent of the weight, of the composition and the hydrocortisone component preferably ranging from about 0.25 percent to about 1 percent by weight, more preferably 0.5 percent by weight, of the composition. It is also preferred that the composition be used in cream form for ease in application and adhesion to the skin surface of the heel.

A preferred heel cream composition is made by mixing (as set out below) three phases, in which are included the active ingredients plus a carrier suitable for topical application to human skin, particularly heels. The following have been found by the inventor to be acceptable ranges, in weight percentage, for the ingredients comprising the heel cream composition:

| Ingredients | Weight Percent |
|---|---|
| For the oil phase | |
| Mineral oil & lanolin alcohol | 4-16 percent |
| Stearyl alcohol & ceteareth-20 | 2.5-10 percent |
| Petrolatum & lanolin & lanolin alcohol | 2-8 percent |
| Mineral oil | 2.25-9 percent |
| Glyceryl stearate | 1-4 percent |
| Vitamin E | 0.5-6 percent |
| Fatty alcohol/polysorbate blend | 1-4 percent |
| For the water phase | |
| Propylene glycol | 2.25-9 percent |
| Germaben 11 | 0.5-2 percent |
| Water | balance |
| For the medicate phase | |
| Salicylic acid | 2-10 percent |
| Hydrocortisone | 0.25-1 percent |

In actual use, application of the heel cream was found to be very effective in eliminating cracks located in the soles, particularly heels, of feet, with complete relief being obtained within a period of approximately two weeks. To facilitate the healing process and thus reduce the total healing time, it is recommended that the dead skin which surrounds epidermal cracks be scraped or removed through the use of a suitable means such as a pumice stone or fine grit sandpaper. To provide a convenient method of disposing of the skin, it is recommended that the scraping procedure be performed while showering or bathing.

Following is an example of a most preferred embodiment of the heel cream for relieving cracks in the heels of feet, prepared as follows, it being specifically noted that the same basic method steps may be applied effectively where the ingredients are within the above-described preferred ranges. In this preferred embodiment, the heel cream was prepared by dividing the substances comprising the heel cream into three separate phases: the oil phase, the water phase, and the medicate phase. The oil phase was prepared by first mixing the ingredients comprising the oil phase together. In preparing this preferred embodiment, the mixing of the ingredients was performed by an homogenation process using a Hamilton kettle (obtainable from Hamilton Kettle Co., located in Cincinnati, Ohio) having a 300 gallon/hour mixing capacity. During this mixing, the combined ingredients comprising the oil phase were heated to a temperature of about 80 degrees Celsius. A preferred formulation of the oil phase composition that was used, given in approximate weight percentage, is:

For the oil phase

| Ingredients | Weight Percent |
|---|---|
| Mineral oil & lanolin alcohol | 8.0 percent |
| Stearyl alcohol & ceteareth-20 | 5.0 percent |
| Petrolatum & lanolin & lanolin alcohol | 4.0 percent |
| Mineral oil | 4.5 percent |
| Glyceryl stearate | 2.0 percent |
| Vitamin E | 3.0 percent |
| Fatty alcohol/polysorbate blend | 2.0 percent |

The ingredients used in the oil phase may be purchased from normal chemical outlets. In the preferred embodiment, Amerchol L-101 was used for the mineral oil and lanolin mix, Amerchol Promulgon D was used the stearyl alcohol and ceteareth-20 mix, and Amerchol CAB was used for the petrolatum and lanolin and lanolin alcohol mix, all available from Amerchol Co., New Jersey. Coroda Polo Wax was used for the fatty alcohol/polysorbate blend, available from Coroda Chemical Co., New York. The mineral oil, glyceryl stearate, and vitamin E are commercially available in most chemical supply outlets.

After the oil phase had been mixed and heated, the water phase was prepared by mixing the water phase ingredients together and simultaneously heating the mixture to a temperature of about 80 degrees Celsius. A preferred formulation of the water phase composition that was used, given in approximate weight percentage, is:

For the water phase

| Ingredients | Weight Percent |
|---|---|
| Water | 56.5 percent |
| Propylene glycol | 4.5 percent |
| Germaben 11 | 1.0 percent |

Of the chemicals used in the water phase, propylene glycol and boric acid are available from normal chemical suppliers. Upon the completion of the water phase attaining a temperature of about 80 degrees Celsius, the oil phase and the water phase were combined and mixed until the combined composition had cooled to a temperature of 45 degrees Celsius (to form a salve mixture). When the combined water phase and oil phase were at 45 degrees Celsius, the medicate phase was added and mixed in to form a final mixture. A preferred formulation of the medicate phase composition that was used, given in approximate weight percentage, is:

For the medicate phase

| Ingredients | Weight Percent |
|---|---|
| Salicylic acid | 4.0 percent |
| Hydrocortisone | 0.5 percent |

The ingredients used in the medicate phase are preferably in powdered form. The hydrocortisone and the salicylic acid used were obtained from Alpha Chemical of Central Islip, N.Y. The final mixture comprising the oil phase, water phase, and medicate phase was then mixed for approximately 15 minutes while cooling, at which stage the formulation was ready for use.

II) Preferred Composition

Effective results were also obtained with the active ingredients preferably comprising between about 4 percent to about 20 percent by weight of the composition, with an acceptable range of the salicylic acid component preferably being between about 2 percent and about 10 percent by weight, more preferably about 4 percent of the weight, of the composition; the hydrocortisone component preferably ranging from about 0.25 percent to about 1 percent by weight, more preferably 0.5 percent by weight, of the composition; the boric acid component preferably ranging from about 1 percent to about 4 percent by weight, most preferably 2 percent by weight, of the composition; and the zinc oxide component preferably ranging from about 1 percent to about 7 percent by weight, most preferably 3 percent by weight, of the composition. It is also preferred that the composition be used in cream form for ease in application and adhesion to the skin surface.

A preferred heel cream composition is made by mixing (as set out below) three phases, in which are included the active ingredients plus a carrier suitable for topical application to human skin. The following have been found by the inventor to be acceptable ranges, in weight percentage, for the ingredients comprising the heel cream composition:

| Ingredients | Weight Percent |
|---|---|
| For the oil phase | |
| Mineral oil & lanolin alcohol | 4-16 percent |
| Stearyl alcohol & ceteareth-20 | 2.5-10 percent |
| Petrolatum & lanolin & lanolin alcohol | 2-8 percent |
| Mineral oil | 2.25-9 percent |
| Glyceryl stearate | 1-4 percent |
| Vitamin E | 0.5-6 percent |
| Fatty alcohol/polysorbate blend | 1-4 percent |
| For the water phase | |
| Propylene glycol | 2.25-9 percent |
| Germaben 11 | 0.5-2 percent |
| Boric acid | 1-4 percent |
| Water | balance |
| For the medicate phase | |
| Salicylic acid | 2-10 percent |
| Hydrocortisone | 0.25-1 percent |
| Zinc Oxide | 1-7 percent |

In actual use, application of the heel cream was found to be very effective in eliminating cracks located in the heels of feet, with complete relief being obtained within a period of approximately two weeks. To facilitate the healing process and thus reduce the total healing time, it is recommended that the dead skin which surrounds epidermal cracks be scraped or removed through the use of a suitable means such as a pumice stone or fine grit sandpaper. To provide a convenient method of disposing of the skin, it is recommended that the scraping procedure be performed while showering or bathing.

Following is another example of a preferred embodiment of the heel cream for relieving cracks in the heels of feet, prepared as follows, it being specifically noted that the same basic method steps may be applied effectively where the ingredients are within the above-described preferred ranges. In this preferred embodiment, the heel cream was prepared by dividing the substances comprising the heel cream into three separate phases: the oil phase, the water phase, and the medicate phase. The oil phase was prepared by first mixing the ingredients comprising the oil phase together. In preparing this preferred embodiment, the mixing of the ingredients was performed by an homogenation process using a Hamilton kettle (obtainable from Hamilton Kettle Co., located in Cincinnati, Ohio) having a 300 gallon/hour mixing capacity. During this mixing, the combined ingredients comprising the oil phase were heated to a temperature of about 80 degrees Celsius. A preferred formulation of the oil phase composition that was used, given in approximate weight percentage, is:

For the oil phase

| Ingredients | Weight Percent |
|---|---|
| Mineral oil & lanolin alcohol | 8.0 percent |
| Stearyl alcohol & ceteareth-20 | 5.0 percent |
| Petrolatum & lanolin & lanolin alcohol | 4.0 percent |
| Mineral oil | 4.5 percent |
| Glyceryl stearate | 2.0 percent |
| Vitamin E | 3.0 percent |
| Fatty alcohol/polysorbate blend | 2.0 percent |

The ingredients used in the oil phase may be purchased from normal chemical outlets. In the preferred embodiment, Amerchol L-101 was used for the mineral oil and lanolin mix, Amerchol Promulgon D was used the stearyl alcohol and ceteareth-20 mix, and Amerchol CAB was used for the petrolatum and lanolin and lanolin alcohol mix, all available from Amerchol Co., New Jersey. Coroda Polo Wax was used for the fatty alcohol/polysorbate blend, available from Coroda Chemical Co., New York. The mineral oil, glyceryl stearate, and vitamin E are commercially available in most chemical supply outlets.

After the oil phase had been mixed and heated, the water phase was prepared by mixing the water phase ingredients together and simultaneously heating the mixture to a temperature of about 80 degrees Celsius. A preferred formulation of the water phase composition that was used, given in approximate weight percentage, is:

For the water phase

| Ingredients | Weight Percent |
|---|---|
| Water | 56.5 percent |
| Propylene glycol | 4.5 percent |
| Germaben 11 | 1.0 percent |
| Boric acid | 2.0 percent |

Of the chemicals used in the water phase, propylene glycol and boric acid are available from normal chemical suppliers. The boric acid used in the example was obtained from U.S. Borax of Valencia, Calif. Upon the completion of the water phase attaining a temperature of about 80 degrees Celsius, the oil phase and the water phase were combined and mixed until the combined composition had cooled to a temperature of 45 degrees Celsius (to form a salve mixture). When the combined water phase and oil phase were at 45 degrees Celsius, the medicate phase was added and mixed in to form a final mixture. A preferred formulation of the medicate phase composition that was used, given in approximate weight percentage, is:

For the medicate phase

| Ingredients | Weight Percent |
|---|---|
| Salicylic acid | 4.0 percent |
| Hydrocortisone | 0.5 percent |
| Zinc oxide | 3.0 percent |

The ingredients used in the medicate phase are preferably in powdered form. The hydrocortisone and the salicylic acid used were obtained from Alpha Chemical of Central Islip, N.Y. The zinc oxide used was obtained from Solvay Fluoride, Inc., of Greenwich, Conn. The final mixture comprising the oil phase, water phase, and medicate phase was then mixed for approximately 15 minutes while cooling, at which stage the formulation was ready for use.

To evidence the usefulness and efficacy of the composition of this invention, numerous trials have been conducted over an extended period of time on those individuals affected by mild to severe foot sole conditions ranging from cracks, calluses and dry skin to the more pronounced and often debilitating condition which is characterized by painful deep fissures or cracks in the heels and balls of the feet (heel cracks). The results of the studies performed in regards to the preferred embodiments of the heel cream composition of the present invention are as hereinafter given.

Trial Study Series A

An ongoing series of trials addressing modifications in the composition of the active ingredients have shown the following results:

### Active Ingredient Study 1

Salicylic acid: Study of salicylic acid compositions in embodiments of the present invention have shown that the effective range of salicylic acid concentration is fairly limited. Too much salicylic acid results in peeling of healthy skin upon the foot and heel, while too little will results in less than effective exfoliation. Concentrations less than about 4% are generally less effective, promoting less exfoliation and having a lengthened healing time. A concentration of about 4% appears to be the most effective concentration although about 5% is also an effective concentration. Salicylic acid concentrations in the range of about 8-9% are not as effective due to excessive exfoliation, particularly near the arch of the foot, and even on the hands if the hands are not well washed following applications of the heal crack healing composition.

### Active Ingredient Study 2

Hydrocortisone: Study of hydrocortisone compositions in embodiments of the present invention have shown that the effective range of hydrocortisone concentration is also fairly limited. Hydrocortisone aids in healing and the soothing of itchy skin and reduction of redness. Concentrations less than about 0.5% are generally less effective, having a lengthened healing time, and concentrations less than about 0.25% have been shown to have little worthwhile effect. Thus, concentrations of about 0.5% or more appear to be the most effective concentrations. It should be noted that although concentrations greater than about 0.75% do promote a faster healing, the increased incremental healing for the concentration increase becomes less. Also, hydrocortisone is an expensive ingredient and the unit cost per incremental increase in hydrocortisone concentration does not have a proportionate incremental increase in healing.

### Active Ingredient Study 3

Zinc oxide: Study of zinc oxide compositions in embodiments of the present invention have shown that the contribution of the zinc oxide is limited in terms of a healing effect. The main contribution of the zinc oxide is as an ingredient to help the active ingredients stick to the affected area. At concentrations above about 5%, the creaminess of the composition starts to decrease, although this does not appear to affect the healing properties of the composition. At compositions having a zinc oxide concentration less than about 1%, some slight decrease in the healing is observed thus requiring a slightly longer time for an equivalent healing. The optimal concentration for healing of cracks upon the heel and foot in some preferred embodiments is about 3% although some preferred embodiments have dispensed entirely with the zinc oxide as an active ingredient with satisfactory results still healing cracks upon the heel.

### Active Ingredient Study 4

Boric acid: Boric acid compositions in embodiments of the present invention are not present for a healing effect, but as a preferred carrier. The boric acid contributes a creamy feel and texture to the heal crack healing composition. Thus some preferred embodiments of the present invention utilize boric acid as a preferred carrier. For the use of boric acid as a carrier, the boric acid component preferably ranges from about 1 percent to about 4 percent by weight, and most preferably 2 percent by weight, of the composition. Additionally, alternative carriers in other preferred embodiments of the composition also used Vaseline, lanolin, etc. as the carrier with satisfactory results. Thus, preferred embodiments of the present invention, a heel crack healing composition and method therefor, may comprise salicylic acid and hydrocortisone as active ingredients, but not necessarily zinc oxide and/or boric acid.

Trial Study Series II

### Trial Study 1

Trial Study 1 was conducted on a group of five individuals (subject nos. 1-5) suffering from a variety of sole ailments comprising calluses, dry skin, and, particularly, heel cracks. These subjects were provided with the heel cream composition having a minimum dose of active ingredients defined herein as about 1 salicylic acid, 0.5% zinc oxide, and about 0.25% hydrocortisone, and directed to apply the cream to the affected areas twice daily. After approximately one month of treatment, all subjects exhibited only mild to moderate improvement. They were then provided with a heel cream composition having a moderate dose of active ingredients defined herein as about 3% salicylic acid, 2% zinc oxide, and about 0.25% hydrocortisone, and directed to continue applying the cream to the affected areas twice daily. Continued mild to moderate improvement was noted with respect the effected foot areas. However, the subjects still exhibited either dry skin and/or small cracks and callused areas after an additional four weeks of treatment, and so all subjects were then provided with this trial's maximum dose of active ingredients defined herein as about 4% salicylic acid, about 3% zinc oxide, and about 0.5% hydrocortisone. After approximately twelve days of treatment with such maximum dose, the skin on all of the subjects feet was soft and smooth and exhibited a pinkish hue evidencing rapid capillary refill. Furthermore, no sign of calluses, cracks/fissures, or dry skin was observed on the feet of the subjects.

### Trial Study 2

Trial Study 2 was conducted on four additional subjects suffering from epidermal heel cracks (subject nos. 6-9) using a formulation of the heel cream composition comprising about 3% salicylic acid and 2% zinc oxide. After approximately two weeks of treatment, the affected areas on the feet exhibited mild to moderate improvement with respect to calluses and dry skin, however no signs of healing of the heel cracks and fissures on two of the subjects was observed. After approximately two further weeks of treatment using the above composition with minimal signs of improvement, a 4% salicylic acid and 3% zinc oxide formulation was provided. An examination of these subjects after approximately 12 days of treatment revealed minimal healing of the cracks, calluses, and dry skin patches. At this point, these subjects were provided with the above-defined maximum dose of active ingredients (about 4% salicylic acid, about 3% zinc oxide, and about 0.5% hydrocortisone). The subjects' feet were examined approximately seven weeks later and all showed soft and smooth skin on the previously affected areas with no signs of cracks, calluses, or dry skin.

### Trial Study 3

Four additional subjects (subject nos. 10-13) with the mentioned typical epidermal heel cracks were selected for Trial Study 3 and were provided with a heel cream composition comprising about 3% salicylic acid and about 0.25% hydrocortisone. After approximately two weeks of treatment, an examination of each patient's feet revealed only a mild improvement. Also, the subjects reported that the composition used felt “watery or slippery” and did not rub into the affected areas very well. Each of the four subjects was then provided with a formulation containing about 4% salicylic acid and 0.5% hydrocortisone and were again examined approximately two weeks later. Although some improvement was noted regarding two of the subjects, the other two subjects evidenced no improvement in the condition of their feet. At this point, each subject was provided with the maximum dose of active ingredients (4% salicylic acid, 3% zinc oxide, and 0.5% hydrocortisone). After approximately eighteen days of treatment with the maximum dose, the skin on all of the subjects feet and toes was soft and smooth and exhibited a pinkish hue evidencing rapid capillary refill.

### Trial Study 4

A clinical study was also conducted under the supervision of a licensed podiatrist using a female subject (subject no. 14) suffering from severe cracking on both feet, particularly heels. The subject was directed to apply the above-defined moderate dose of active ingredients (see Trial Study 1) to the affected areas of only one foot twice daily. The treated foot evidenced significant improvement in only one week. After approximately one month of treatment using the moderate dose, the subject was directed to begin treatment using the above-defined maximum (see Trial Study 1) dose. After approximately ten days of treatment, no cracks, fissures, or thick calluses remained on the subject's treated foot.

### Trial Study 5

Four subjects (subject nos. 15-18) were provided with the above-defined maximum dose of active ingredients (4% salicylic acid, 3% zinc oxide, and 0.5% hydrocortisone) and instructed to apply the composition to their respective feet twice daily. After approximately three weeks of treatment, all four subjects reported superb results with complete healing being observed for those patients having deep epidermal heel cracks, calluses, and dry skin.

### Trial Study 6

Six subjects (subject nos. 19-24) were selected for trial study 6 and divided into two groups. The first group consisted of three of the subjects (19-21) who were provided with a heel cream formulation comprising 5% salicylic acid, 3% zinc oxide, and 0.5% hydrocortisone; the remaining three subjects (22-24) comprised the second group and were provided with a heel cream formulation comprising 6% salicylic acid, 3% zinc oxide, and 0.5% hydrocortisone. Both groups were directed to apply their respective formulations twice daily. After approximately two weeks of treatment, the cracks, fissures, and calluses on the feet of all six subjects were completely gone. However, two of the subjects who continued to use the 6% salicylic acid formulation reported peeling of the skin on the fingers/hand used to apply the composition.

During the course of the above studies it has been observed that optimal results can be achieved with the heel cream composition described herein having a salicylic acid component most preferably comprising about 4% by weight of the composition, a zinc oxide component most preferably comprising about 3% by weight of the composition, and a hydrocortisone component most preferably comprising about 0.5% by weight of the composition. Furthermore, it has been observed that an increase in the salicylic acid component to over about 6% or 7% by weight begins to yield negative results characterized not only by a rapid defoliation of the affected areas—thus preventing the restoration of a new layer of smooth skin over these areas—but also defoliation of the skin on other body parts in frequent contact with the composition. At about 10% by weight salicylic acid, the effects of rapid defoliation begin to exceed the therapeutic benefits derived from the composition.

According to the preferred embodiments of the present invention, the best results have been obtained by rubbing the heel cream onto the affected areas twice daily; once in the morning after bathing, and again at night prior to bedtime. This process should be repeated until the epidermal cracks have disappeared. Care should be taken to wash the hands thoroughly after applying the heel cream. To facilitate the healing process, it is recommended that an abrasive material such as fine grit sandpaper or a pumice stone be used to exfoliate or scrape the dead skin surrounding the cracks. To easier dispose of the dead skin, it is also recommended that this scraping process be performed in the shower, thereby allowing the dead skin to simply be flushed down the drain. Once complete healing of the affected areas of the foot or feet has been achieved, the individual may continue to apply the heel cream once daily to prevent reformation of cracks, fissures, dry skin, or calluses.

Although the preferred embodiments of this invention have been described herein, it will be understood that the broadest scope of this invention includes any changes which may be made within the scope of what is claimed hereinafter without departing from the spirit of the invention. Further, many other advantages of the present invention will be apparent to those skilled in the art from the above description and the below claims.

What is claimed is:

1. A method for healing epidermal cracks in heels comprising the acts of:
a) topically applying to the affected skin areas having said epidermal cracks a composition having a safe and effective amount of active ingredients consisting essentially of salicylic acid and hydrocortisone, mixed with a suitable cream mixture carrier;
b) applying said composition to the affected skin areas at least once daily to treat heel cracks; and
c) removing the dead skin surrounding the epidermal cracks with an abrasive material;
d) wherein said suitable cream mixture carrier is a member of the class of carriers having properties substantially comparable to the carrier properties of Vaseline and lanolin.

2. The method according to claim 1 wherein said active ingredients of said composition consist essentially of:
a) from about 2 percent to about 10 percent by weight of said composition of salicylic acid; and
b) from about 0.25 percent to about 1 percent by weight of said composition of hydrocortisone.

3. The method according to claim 1 wherein said active ingredients of said composition consist essentially of:
a) about 4 percent by weight of said composition of salicylic acid; and
b) about 0.5 percent by weight of said composition of hydrocortisone.

4. The method of claim 1 wherein said application of said composition is done at least twice daily until the epidermal cracks heal.

5. The method of claim 1 further comprising the subsequent step of applying said composition to the affected skin areas at least once daily to maintain the heels in a healed condition.

6. The method of claim 1 wherein said abrasive material comprises a pumice stone.

7. The method of claim 1 wherein said abrasive material comprises fine grit sandpaper.

8. A composition, for treating and healing epidermal cracks on heels, consisting essentially of the following ingredients in the following amounts, by approximate weight percentage of said composition:

| Ingredients | Weight Percent |
|---|---|
| Mineral oil & lanolin alcohol | 8 percent |
| Stearyl alcohol & ceteareth-20 | 5 percent |
| Petrolatum & lanolin & lanolin alcohol | 4 percent |
| Mineral oil | 4.5 percent |
| Glyceryl stearate | 2 percent |
| Vitamin E | 3 percent |
| Fatty alcohol/polysorbate blend | 2 percent |
| Propylene glycol | 4.5 percent |
| Preservative | 1 percent |
| Salicylic acid | 4 percent |
| Hydrocortisone | 0.5 percent |
| Water | balance |

9. A method for healing epidermal cracks in heels comprising the acts of:
a) topically applying to the affected skin areas having said epidermal cracks a composition having a safe and effective amount of active ingredients consisting essentially of salicylic acid, hydrocortisone, and zinc oxide, mixed with a suitable cream mixture carrier;
b) applying said composition to the affected skin areas at least once daily to treat heel cracks; and
c) removing the dead skin surrounding the epidermal cracks with an abrasive material.

10. The method according to claim 9 wherein said active ingredients of said composition consist essentially of:
a) from about 2 percent to about 10 percent by weight of said composition of salicylic acid;
b) from about 0.25 percent to about 1 percent by weight of said composition of hydrocortisone;
c) from about 1 percent to about 4 percent by weight of said composition of boric acid; and
d) from about 1 percent to about 7 percent by weight of said composition of zinc oxide.

11. The method according to claim 10 wherein said active ingredients of said composition consist essentially of:
a) about 4 percent by weight of said composition of salicylic acid;
b) about 0.5 percent by weight of said composition of hydrocortisone;
c) about 2 percent by weight of said composition of boric acid; and
d) about 3 percent by weight of said composition of zinc oxide.

12. The method of claim 9 wherein said application of said composition is done at least twice daily until the epidermal cracks heal.

13. The method of claim 12 further comprising the subsequent step of applying said composition to the affected skin areas at least once daily to maintain the feet in a healed condition.

14. The method of claim 9 wherein said abrasive material comprises a pumice stone.

15. The method of claim 9 wherein said abrasive material comprises fine grit sandpaper.

16. A composition, for treating and healing epidermal cracks on heels, consisting essentially of the following ingredients in the following amounts, by approximate weight percentage of said composition:

| Ingredients | Weight Percent |
|---|---|
| Mineral oil & lanolin alcohol | 8 percent |
| Stearyl alcohol & ceteareth-20 | 5 percent |
| Petrolatum & lanolin & lanolin alcohol | 4 percent |
| Mineral oil | 4.5 percent |
| Glyceryl stearate | 2 percent |
| Vitamin E | 3 percent |
| Fatty alcohol/polysorbate blend | 2 percent |
| Propylene glycol | 4.5 percent |
| Preservative | 1 percent |
| Boric acid | 2 percent |
| Salicylic acid | 4 percent |
| Hydrocortisone | 0.5 percent |
| Zinc Oxide | 3 percent |
| Water | 56.5 percent |
